(19) United States
(12) Patent Application Publication
(10) Pub. No.: US 2024/0379203 A1
(43) Pub. Date: Nov. 14, 2024

(54) INFORMATION PROCESSING APPARATUS, METHOD, AND PROGRAM

(71) Applicant: FUJIFILM CORPORATION, Tokyo (JP)

(72) Inventor: Akimichi ICHINOSE, Tokyo (JP)

(21) Appl. No.: 18/657,644

(22) Filed: May 7, 2024

(30) Foreign Application Priority Data
May 10, 2023 (JP) 2023-078181

(51) Int. Cl. G16H 15/00

(52) U.S. Cl. CPC G16H 15/00

(57) ABSTRACT

A processor is configured to: acquire a plurality of medical documents having different creation times; specify mutually corresponding descriptions in each of one medical document among the plurality of medical documents and at least one other medical document other than the one medical document; display the one medical document on a display; receive designation of a character string for the one medical document; and display a description of the at least one other medical document corresponding to a description including the designated character string in a manner different from other descriptions other than the description corresponding to the description including the designated character string.

### CROSS REFERENCE TO RELATED APPLICATIONS

The present application claims priority from Japanese Patent Application No. 2023-078181, filed on May 10, 2023, the entire disclosure of which is incorporated herein by reference.

### BACKGROUND

### Technical Field

The present disclosure relates to an information processing apparatus, method, and program.

### Related Art

In recent years, advances in medical devices, such as computed tomography (CT) apparatuses and magnetic resonance imaging (MRI) apparatuses, have enabled image diagnosis using high-resolution medical images with higher quality. In particular, in a case of performing image diagnosis using CT images, MRI images, or the like, an interpretation report including an interpretation result of an image is created. Various methods have been proposed to support the creation of medical documents such as interpretation reports.

For example, JP2019-153249A proposes a method of acquiring the latest current medical image of a patient and a past interpretation report of the same patient, specifying characters included in the interpretation report corresponding to a designated position in the displayed current medical image, and emphasizing and displaying the specified characters. According to the method described in JP2019-153249A, since a user can easily refer to a description of the past interpretation report related to the current medical image, the user can easily create an interpretation report for the current medical image.

However, the method described in JP2019-153249A cannot compare a current interpretation report created for a current medical image with a past interpretation report.

### SUMMARY OF THE INVENTION

The present disclosure has been made in view of the above circumstances, and an object of the present disclosure is to make it possible to easily compare medical documents such as interpretation reports having different creation times.

According to an aspect of the present disclosure, there is provided an information processing apparatus comprising: at least one processor, in which the processor is configured to: acquire a plurality of medical documents having different creation times; specify mutually corresponding descriptions in each of one medical document among the plurality of medical documents and at least one other medical document other than the one medical document; display the one medical document on a display; receive designation of a character string for the one medical document; and display a description of the at least one other medical document corresponding to a description including the designated character string in a manner different from other descriptions other than the description corresponding to the description including the designated character string.

In the information processing apparatus according to the aspect of the present disclosure, the processor may be configured to: classify descriptions included in each of the plurality of medical documents for each lesion; and specify the mutually corresponding descriptions for each lesion.

In the information processing apparatus according to the aspect of the present disclosure, the processor may be configured to: further specify a difference between corresponding descriptions included in each of the plurality of medical documents; and display the difference in a manner different from descriptions other than the difference in the at least one other medical document, instead of the description corresponding to the description including the designated character string or in addition to the description corresponding to the description including the designated character string.

In the information processing apparatus according to the aspect of the present disclosure, the medical document may include a medical image corresponding to the medical document, and the processor may be configured to display at least a partial region of the medical image related to the differently displayed description in a manner different from other regions other than the partial region.

In the information processing apparatus according to the aspect of the present disclosure, the processor may be configured to display character strings related to the mutually corresponding descriptions in the one medical document in a manner different from other character strings other than the related character strings.

In the information processing apparatus according to the aspect of the present disclosure, the processor may be configured to display the description including the character string in the one medical document in a manner different from other descriptions other than the description including the character string.

In the information processing apparatus according to the aspect of the present disclosure, the one medical document may be a medical document currently being created.

In the information processing apparatus according to the aspect of the present disclosure, the plurality of medical documents may include medical documents acquired by different modalities at least in part.

In the information processing apparatus according to the aspect of the present disclosure, the processor may be configured to, in a case in which a plurality of the other medical documents are displayed, preferentially display the description in the different manner for the other medical document whose creation time is closer to the one medical document.

According to another aspect of the present disclosure, there is provided an information processing method executed by a computer, the information processing method comprising: acquiring a plurality of medical documents having different creation times; specifying mutually corresponding descriptions in each of one medical document among the plurality of medical documents and at least one other medical document other than the one medical document; displaying the one medical document on a display; receiving designation of a character string for the one medical document; and displaying a description of the at least one other medical document corresponding to a description including the designated character string in a manner different from other descriptions other than the description corresponding to the description including the designated character string.

According to another aspect of the present disclosure, there is provided an information processing program causing a computer to execute: acquiring a plurality of medical documents having different creation times; specifying mutually corresponding descriptions in each of one medical document among the plurality of medical documents and at least one other medical document other than the one medical document; displaying the one medical document on a display; receiving designation of a character string for the one medical document; and displaying a description of the at least one other medical document corresponding to a description including the designated character string in a manner different from other descriptions other than the description corresponding to the description including the designated character string.

According to the aspects of the present disclosure, it is possible to easily compare medical documents such as interpretation reports having different creation times.

### BRIEF DESCRIPTION OF THE DRAWINGS

FIG. 1 is a diagram showing a schematic configuration of a medical information system to which an information processing apparatus according to a first embodiment of the present disclosure is applied.

FIG. 2 is a diagram showing a schematic configuration of the information processing apparatus according to the first embodiment.

FIG. 3 is a functional configuration diagram of the information processing apparatus according to the first embodiment.

FIG. 4 is a diagram for describing specification of a description in the first embodiment.

FIG. 5 is a diagram showing a display screen of an interpretation report in the first embodiment.

FIG. 6 is a diagram showing the display screen of the interpretation report in the first embodiment.

FIG. 7 is a flowchart showing a process performed in the first embodiment.

FIG. 8 is a diagram showing another example of the display screen of the interpretation report in the first embodiment.

FIG. 9 is a diagram showing a creation screen of an interpretation report in a second embodiment.

FIG. 10 is a diagram showing a display screen of an interpretation report in a third embodiment.

FIG. 11 is a diagram for describing specification of a description and a difference in a fourth embodiment.

FIG. 12 is a diagram showing a display screen of an interpretation report in the fourth embodiment.

FIG. 13 is a diagram showing another example of the display screen of the interpretation report in the fourth embodiment.

FIG. 14 is a diagram showing a display screen of a plurality of interpretation reports.

FIG. 15 is a diagram showing another example of a display screen of a plurality of interpretation reports.

FIG. 16 is a diagram showing another example of a display screen of an interpretation report in the first embodiment.

FIG. 17 is a diagram showing another example of a display screen of an interpretation report in the first embodiment.

### DETAILED DESCRIPTION

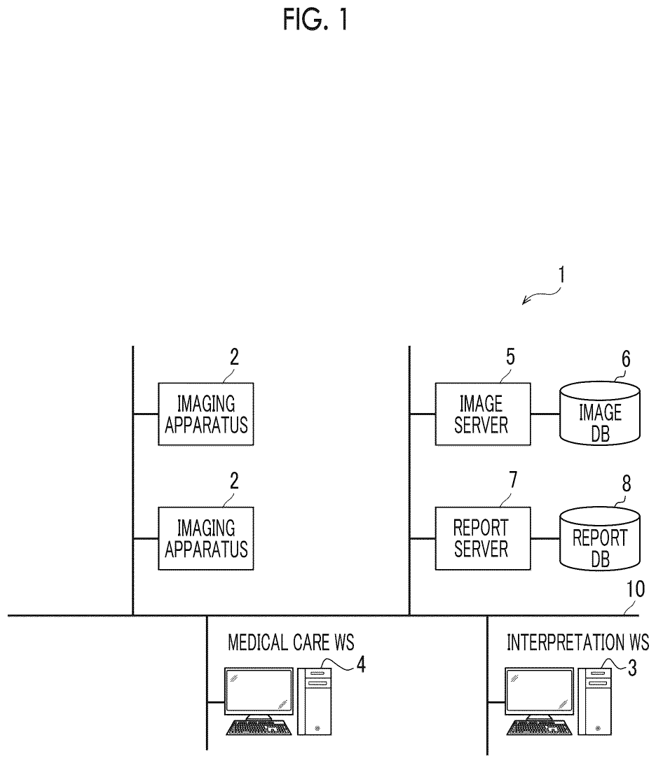

Hereinafter, embodiments of the present disclosure will be described with reference to the drawings. First, a configuration of a medical information system 1 to which an information processing apparatus according to the present embodiment is applied will be described. FIG. 1 is a diagram showing a schematic configuration of the medical information system 1. The medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source.

As shown in FIG. 1, in the medical information system 1, a plurality of imaging apparatuses 2, a plurality of interpretation workstations (WSs) 3 that are interpretation terminals, a medical care WS 4, an image server 5, an image database (hereinafter referred to as an image DB) 6, a report server 7, and a report database (hereinafter referred to as a report DB) 8 are communicably connected to each other through a wired or wireless network 10.

Each apparatus is a computer on which an application program for causing each apparatus to function as a component of the medical information system 1 is installed. The application program is recorded on a recording medium, such as a digital versatile disc (DVD) or a compact disc read-only memory (CD-ROM), and distributed, and is installed on the computer from the recording medium. Alternatively, the application program is stored in a storage apparatus of a server computer connected to the network 10 or in a network storage in a state in which it can be accessed from the outside, and is downloaded to and installed on the computer in response to a request.

The imaging apparatus 2 is an apparatus (modality) that generates a medical image showing a diagnosis target part of the subject by imaging the diagnosis target part. Specifically, examples of the imaging apparatus include a simple X-ray imaging apparatus, a CT apparatus, an MRI apparatus, a positron emission tomography (PET) apparatus, and the like. The medical image generated by the imaging apparatus 2 is transmitted to the image server 5 and is stored in the image DB 6.

The interpretation WS 3 is a computer used by, for example, a radiologist of a radiology department to interpret a medical image and to create an interpretation report, and encompasses an information processing apparatus 20 according to the present embodiment. In the interpretation WS 3, a viewing request for a medical image to the image server 5, various image processing for the medical image received from the image server 5, display of the medical image, input reception of comments on findings regarding the medical image, and the like are performed. In the interpretation WS 3, an analysis process for medical images and input comments on findings, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by a doctor in the medical department for detailed observation of images, viewing of interpretation reports, creation of electronic medical records, and the like, and encompasses the information processing apparatus 20 according to the present embodiment. In the medical care WS 4, a viewing request for the image to the image server 5, display of the image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 comprises a storage in which the image DB 6 is configured. This storage may be a hard disk apparatus connected to the image server 5 by a data bus, or may be a disk apparatus connected to a storage area network (SAN) or a network attached storage (NAS) connected to the network 10. In a case in which the image server 5 receives a request to register a medical image from the imaging apparatus 2, the image server 5 prepares the medical image in a format for a database and registers the medical image in the image DB 6. In the present embodiment, it is assumed that a diagnostic guideline according to the disease is also stored in the image server 5, but the present disclosure is not limited thereto.

Image data of the medical image acquired by the imaging apparatus 2 and accessory information are registered in the image DB 6. The accessory information includes, for example, an image identification (ID) for identifying each medical image, a patient ID for identifying a subject, an examination ID for identifying an examination, a unique ID (unique identification (UID)) allocated for each medical image, examination date and examination time at which a medical image is generated, the type of imaging apparatus used in an examination for acquiring a medical image, patient information such as the name, age, and gender of a patient, an examination part (an imaging part), imaging information (an imaging protocol, an imaging sequence, an imaging method, imaging conditions, the use of a contrast medium, and the like), and information such as a series number or a collection number in a case in which a plurality of medical images are acquired in one examination.

In addition, in a case in which the viewing request from the interpretation WS 3 and the medical care WS 4 is received through the network 10, the image server 5 searches for a medical image registered in the image DB 6 and transmits the searched for medical image to the interpretation WS 3 and to the medical care WS 4 that are request sources.

The report server 7 incorporates a software program for providing a function of a database management system to a general-purpose computer. In a case in which the report server 7 receives a request to register the interpretation report from the interpretation WS 3, the report server 7 prepares the interpretation report in a format for a database and registers the interpretation report in the report DB 8.

In the report DB 8, an interpretation report including at least the comments on findings created by the radiologist using the interpretation WS 3 is registered. The interpretation report may include, for example, information such as a medical image to be interpreted, an image ID for identifying the medical image, a radiologist ID for identifying the radiologist who performed the interpretation, a lesion name, lesion position information, information for accessing a medical image including a specific region, and property information.

Further, in a case in which the report server 7 receives the viewing request for the interpretation report from the interpretation WS 3 and the medical care WS 4 through the network 10, the report server 7 searches for the interpretation report registered in the report DB 8, and transmits the searched for interpretation report to the interpretation WS 3 and to the medical care WS 4 that are request sources.

In the present embodiment, it is assumed that the medical image is a three-dimensional CT image consisting of a plurality of tomographic images with a lung as a diagnosis target, and an interpretation report on an abnormal shadow included in the lung is created by interpreting the CT image in the interpretation WS 3. The medical image is not limited to the CT image, and any medical image such as an MRI image and a simple two-dimensional image acquired by a simple X-ray imaging apparatus can be used.

The network 10 is a wired or wireless local area network that connects various apparatuses in a hospital to each other. In a case in which the interpretation WS 3 is installed in another hospital or clinic, the network 10 may be configured to connect local area networks of respective hospitals through the Internet or a dedicated line.

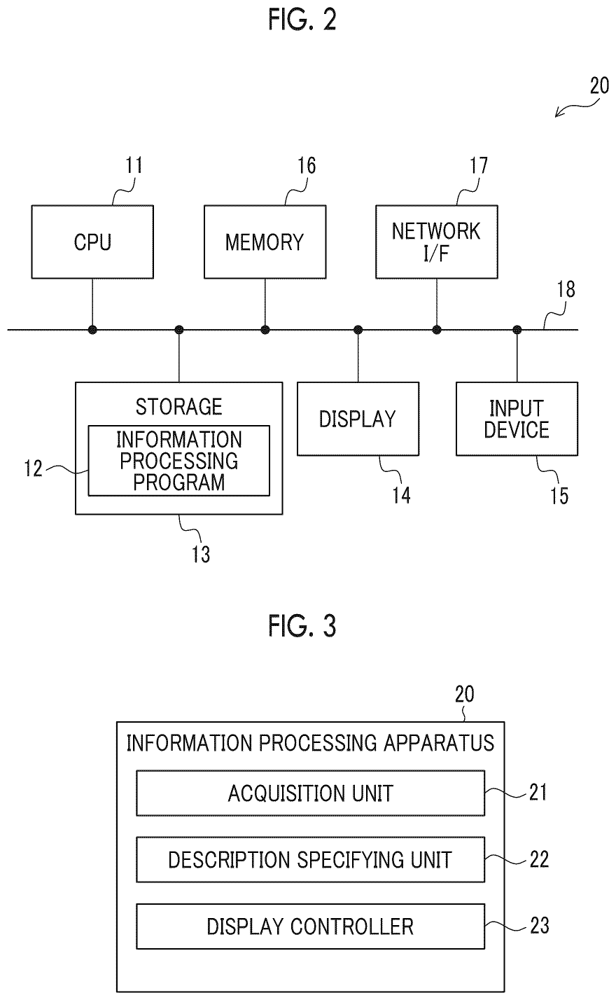

Next, the information processing apparatus according to the first embodiment of the present disclosure will be described. FIG. 2 describes a hardware configuration of the information processing apparatus according to the first embodiment. As shown in FIG. 2, the information processing apparatus 20 includes a central processing unit (CPU) 11, a non-volatile storage 13, and a memory 16 as a temporary storage area. Further, the information processing apparatus 20 includes a display 14 such as a liquid crystal display, an input device 15 such as a keyboard and a mouse, and a network interface (I/F) 17 connected to the network 10. The CPU 11, the storage 13, the display 14, the input device 15, the memory 16, and the network I/F 17 are connected to a bus 18. The CPU 11 is an example of a processor in the present disclosure.

The storage 13 is realized by a hard disk drive (HDD), a solid-state drive (SSD), a flash memory, and the like. An information processing program 12 is stored in the storage 13 as the storage medium. The CPU 11 reads out the information processing program from the storage 13, loads the read information processing program into the memory 16, and executes the loaded information processing program 12.

Next, a functional configuration of the information processing apparatus according to the first embodiment will be described. FIG. 3 is a diagram showing a functional configuration of the information processing apparatus according to the first embodiment. As shown in FIG. 3, the information processing apparatus 20 according to the first embodiment comprises an acquisition unit 21, a description specifying unit 22, and a display controller 23. Then, in a case in which the CPU 11 executes the information processing program, the CPU 11 functions as the acquisition unit 21, the description specifying unit 22, and the display controller 23. In the following, an information processing apparatus encompassed in the medical care WS 4 will be described, but the present disclosure is not limited thereto.

The acquisition unit 21 acquires an interpretation report on the patient to be examined from the report DB 8 in response to an instruction by a user such as a doctor from the input device 15. An interpretation report is an example of a medical document in the present disclosure. Here, the interpretation report to be acquired is a current interpretation report R1 to be diagnosed from now on, and at least one past interpretation report Rpi (i=1 to n) created chronologically earlier than the current interpretation report. The interpretation reports R1 and Rpi acquired by the acquisition unit 21 are stored in the storage 13. Note that the acquisition unit 21 may acquire, from the image DB 6, the medical image for which the current interpretation report R1 has been created for the patient.

The description specifying unit 22 analyzes the interpretation reports R1 and Rpi acquired by the acquisition unit 21, thereby specifying mutually corresponding descriptions included in each of the interpretation reports R1 and Rpi. Specifically, the description specifying unit 22 analyzes the interpretation reports R1 and Rpi using natural language processing technology, thereby classifying the sentences included in the interpretation reports R1 and Rpi into the sentences for each lesion. Further, the description specifying unit 22 may specify, as a keyword, a character string representing the lesion included in the sentence for each lesion.

The natural language processing is a series of technologies for causing a computer to process a natural language that humans use on a daily basis. By the natural language processing, it is possible to divide a sentence into words, analyze the syntax, analyze the meaning, and the like.

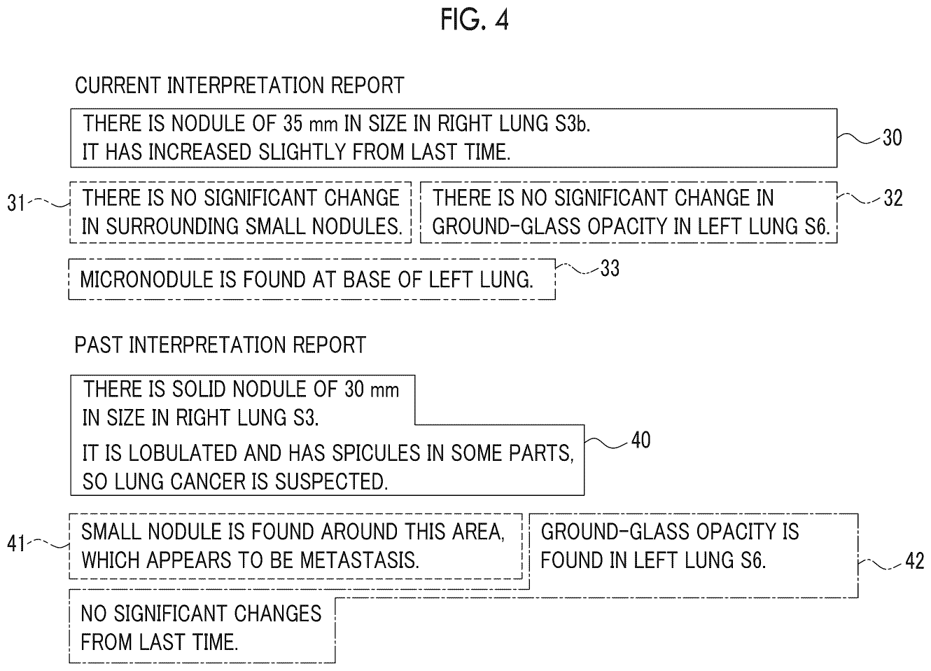

FIG. 4 is a diagram for describing specification of a description. Here, for the sake of description, it is assumed that there is only one past interpretation report. As shown in FIG. 4, in the current interpretation report R1, “There is a nodule of 35 mm in size in right lung S3b. It has increased slightly from last time. There is no significant change in the surrounding small nodules. There is no significant change in the ground-glass opacity in left lung S6. A micronodule is found at the base of the left lung” are described. In the past interpretation report Rp1, “There is a solid nodule of 30 mm in size in right lung S3. It is lobulated and has spicules in some parts, so lung cancer is suspected. A small nodule is found around this area, which appears to be metastasis. A ground-glass opacity is found in left lung S6. No significant changes from last time” are described.

The description specifying unit 22 specifies a description regarding the lesion included in the sentences of the interpretation reports R1 and Rpi. In the present embodiment, the description specifying unit 22 discriminates the description of the lesion using natural language processing. Then, the sentences are structured by classifying the sentences described in the interpretation reports R1 and Rpi for each discriminated lesion.

For example, the description specifying unit 22 structures the current interpretation report R1 shown in FIG. 4 into four sentences 30 to 33, such as “There is a nodule of 35 mm in size in right lung S3b. It has increased slightly from last time” (referred to as a sentence 30), which a sentence regarding the nodule, “There is no significant change in the surrounding small nodules” (referred to as a sentence 31), which is a sentence regarding the small nodule, “There is no significant change in the ground-glass opacity in left lung S6” (referred to as a sentence 32), which is a sentence regarding the ground-glass opacity, and “A micronodule is found at the base of the left lung” (referred to as a sentence 33), which is a sentence regarding the micronodule.

In addition, the description specifying unit 22 structures the past interpretation report Rp1 into three sentences 40 to 42, such as “There is a solid nodule of 30 mm in size in right lung S3. It is lobulated and has spicules in some parts, so lung cancer is suspected” (referred to as a sentence 40), which is a sentence regarding the nodule, “A small nodule is found around this area, which appears to be metastasis” (referred to as a sentence 41), which is a sentence regarding the small nodule, and “A ground-glass opacity is found in left lung S6. No significant changes from last time” (referred to as a sentence 42), which is a sentence regarding the ground-glass opacity.

The description specifying unit 22 specifies mutually corresponding descriptions included in each of the current interpretation report R1 and the past interpretation report Rpi by associating the sentences regarding the same lesion among the four sentences 30 to 33 obtained by structuring the current interpretation report R1 and the three sentences 40 to 42 obtained by structuring the past interpretation report Rpi. Accordingly, the sentence 30 regarding the nodule of the current interpretation report R1 and the sentence 40 regarding the nodule of the past interpretation report Rpi are specified as corresponding descriptions. Further, the sentence 31 regarding the small nodule of the current interpretation report R1 and the sentence 41 regarding the small nodule of the past interpretation report Rpi are specified as corresponding descriptions. Further, the sentence 32 regarding the ground-glass opacity of the current interpretation report R1 and the sentence 42 regarding the ground-glass opacity of the past interpretation report Rpi are specified as corresponding descriptions.

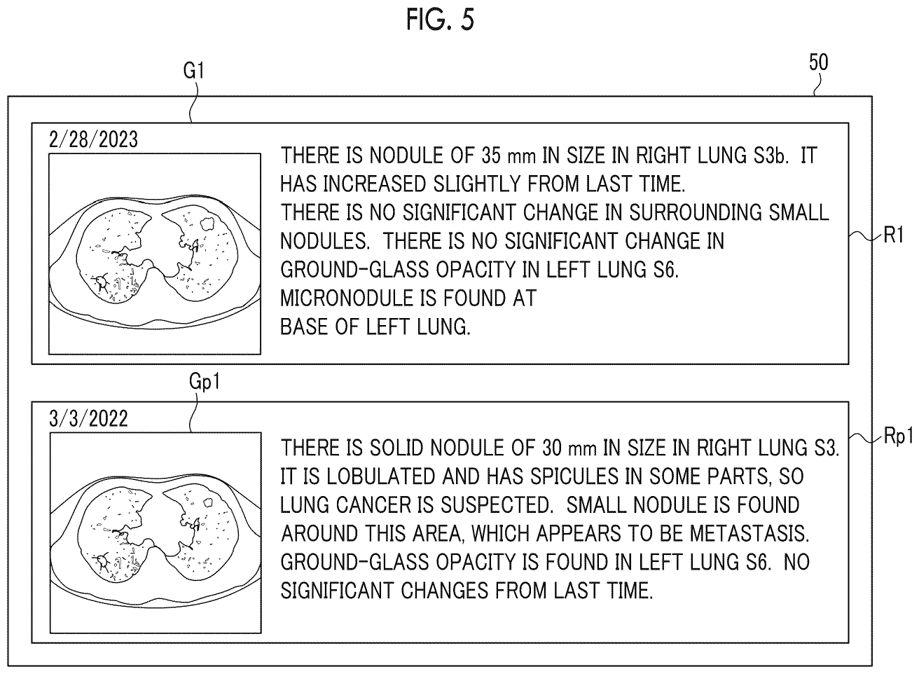

The display controller 23 displays the current interpretation report R1 and the past interpretation report Rpi acquired by the acquisition unit 21 on the display 14. FIG. 5 is a diagram showing a display screen of an interpretation report. As shown in FIG. 5, the current interpretation report R1 and the past interpretation report Rp1 are displayed on a display screen 50 of the interpretation report. The creation date and time of the current interpretation report R1 is Feb. 28, 2023, and the creation date and time of the past interpretation report Rp1 is Mar. 3, 2022. Furthermore, the current interpretation report R1 and the past interpretation report Rp1 each include medical images G1 and Gp1 for which the interpretation report is created.

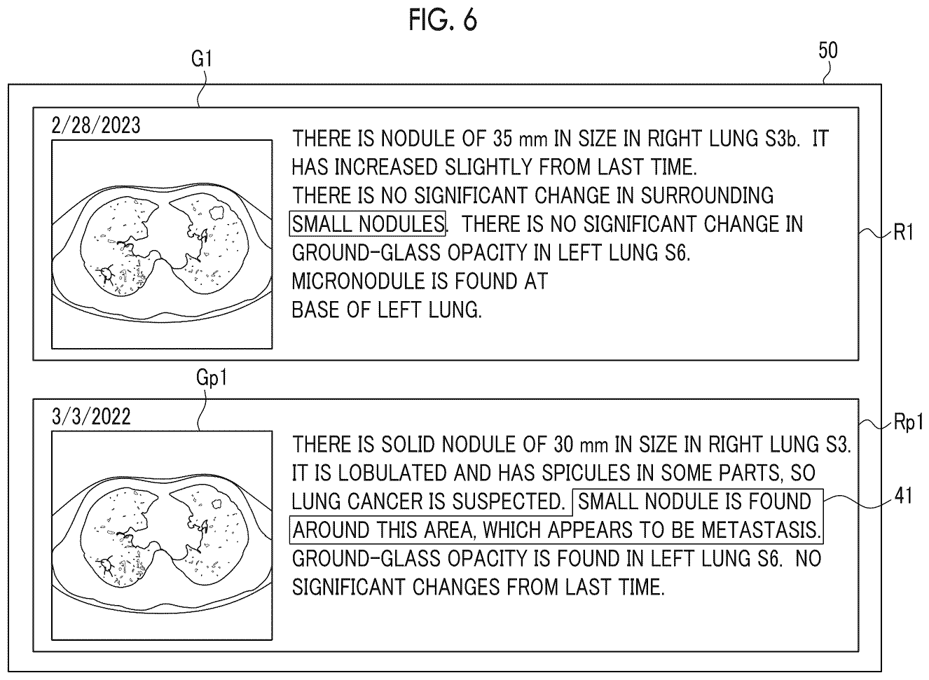

The user can select a character string by moving a cursor using the input device 15 in the description of the current interpretation report R1. For example, as shown in FIG. 6, it is assumed that the user selects “small nodules” described in the current interpretation report R1. In FIG. 6, the selection of “small nodules” is represented by being surrounded with a rectangle. The display controller 23 displays, in the past interpretation report Rp1, the sentence 41 whose description corresponds to the sentence 31 in which “small nodules” is described in the current interpretation report R1 in a manner different from other sentences other than the sentence 41. Specifically, the sentence 41 is highlighted. Any method can be used for highlighting, such as adding a color to the background of the sentence 41, adding an underline, changing the font color, and changing the font, for example. Furthermore, the sentence 41 may be displayed in a manner different from other sentences other than the sentence 41, such as by making the color of the characters of other sentences other than the sentence 41 lighter, or by painting other sentences other than the sentence 41 black. In FIG. 6, the sentence 41 is surrounded with a rectangle to show that the sentence 41 is highlighted. Note that in a case in which “nodule” described in the current interpretation report R1 is designated, the sentence 40 in the past interpretation report Rp1 is highlighted. Furthermore, in a case in which the “ground-glass opacity” described in the current interpretation report R1 is designated, the sentence 42 in the past interpretation report Rp1 is highlighted.

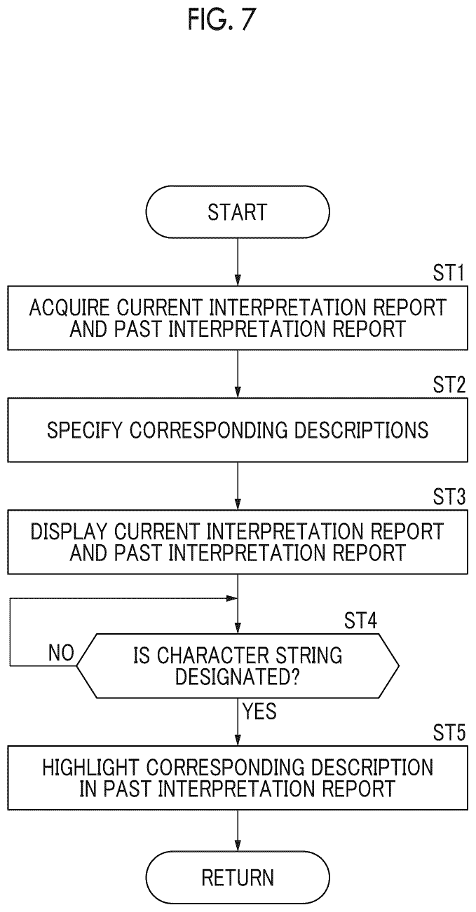

Next, a process performed in the first embodiment will be described. FIG. 7 is a flowchart showing the process performed in the first embodiment. First, the acquisition unit 21 acquires a plurality of medical documents having different creation times, that is, the current interpretation report R1 and the past interpretation report Rpi, from the report DB 8 (Step ST1). Next, the description specifying unit 22 specifies mutually corresponding descriptions included in each of the current interpretation report R1 and the past interpretation report Rpi (Step ST2). Then, the display controller 23 displays the current interpretation report R1 and the past interpretation report Rpi on the display 14 (Step ST3).

Subsequently, monitoring of whether or not the character string is designated in the current interpretation report R1 is started (Step ST4), and in a case in which determination in Step ST4 is affirmative, the display controller 23 displays the sentence of the description corresponding to the description including the designated character string in a manner different from other descriptions other than the description corresponding to the description including the designated character string, that is, highlights it (Step ST5), and returns to Step ST4.

In this way, in the first embodiment, the designation of a character string for the current interpretation report R1 is received, and the description of the past interpretation report Rpi corresponding to the description including the designated character string is displayed in a manner different from other descriptions other than the description corresponding to the description including the designated character string. Therefore, it is possible to easily compare medical documents such as interpretation reports having different creation times.

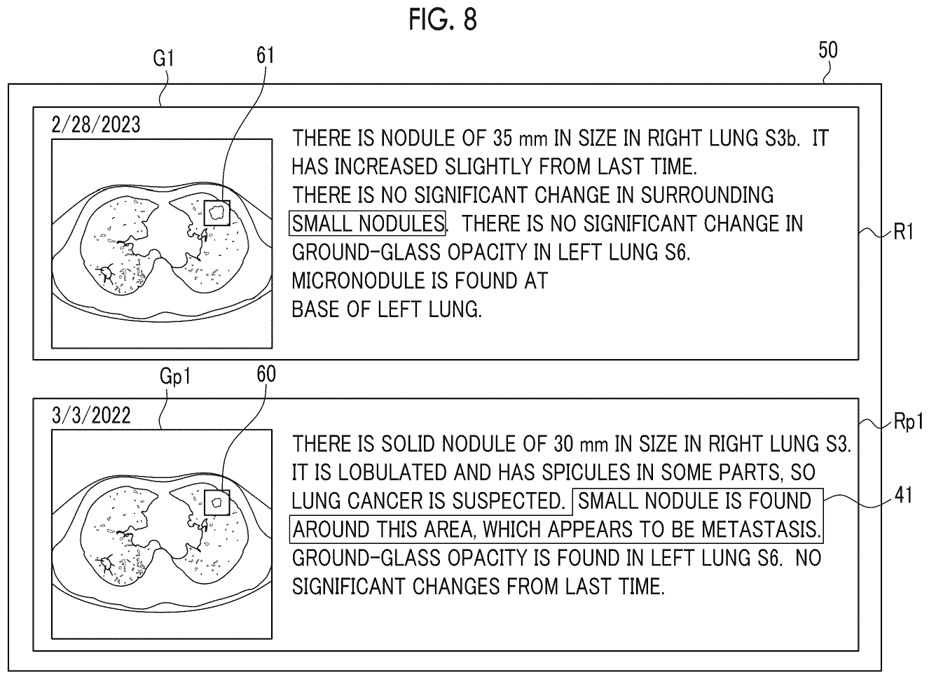

In addition, in the first embodiment, in the medical image Gp1 included in the past interpretation report Rpi, a partial region including at least a lesion included in a sentence displayed in a different manner may be displayed in a manner different from other regions other than the partial region. Specifically, the partial region may be highlighted. Further, a partial region including at least a lesion corresponding to the selected character string included in the current interpretation report R1 may be highlighted. For example, as shown in FIG. 8, a partial region including a lesion may be highlighted by surrounding the lesion with a marker 60 in the medical image Gp1 included in the past interpretation report Rpi. Furthermore, a partial region including a lesion may be highlighted by surrounding the lesion with a marker 61 in the medical image G1 included in the current interpretation report R1. Note that instead of the rectangular markers 60 and 61, arrows or the like may be added.

Further, in the first embodiment, the description specifying unit 22 may specify character strings representing lesions included in the structured sentences 30 to 33 in the current interpretation report R1, and add a link to a sentence including a corresponding description in the past interpretation report Rpi for the specified character string. Accordingly, in a case in which the user uses the input device 15 to click a character string representing a lesion included in the current interpretation report R1, the display controller 23 can display the sentence linked to the clicked character string in the past interpretation report Rpi in a manner different from other sentences other than the linked sentence.

In addition, in the first embodiment, the current interpretation report R1 and the past interpretation report Rpi are acquired at the same time, but the present disclosure is not limited thereto. Even in a case in which the information processing apparatus 20 according to the present embodiment is applied to the interpretation WS 3 to create an interpretation report for a medical image while referring to the display of the past interpretation report Rpi in the interpretation WS 3, the information processing apparatus 20 according to the present embodiment can be applied. This will be described below as a second embodiment.

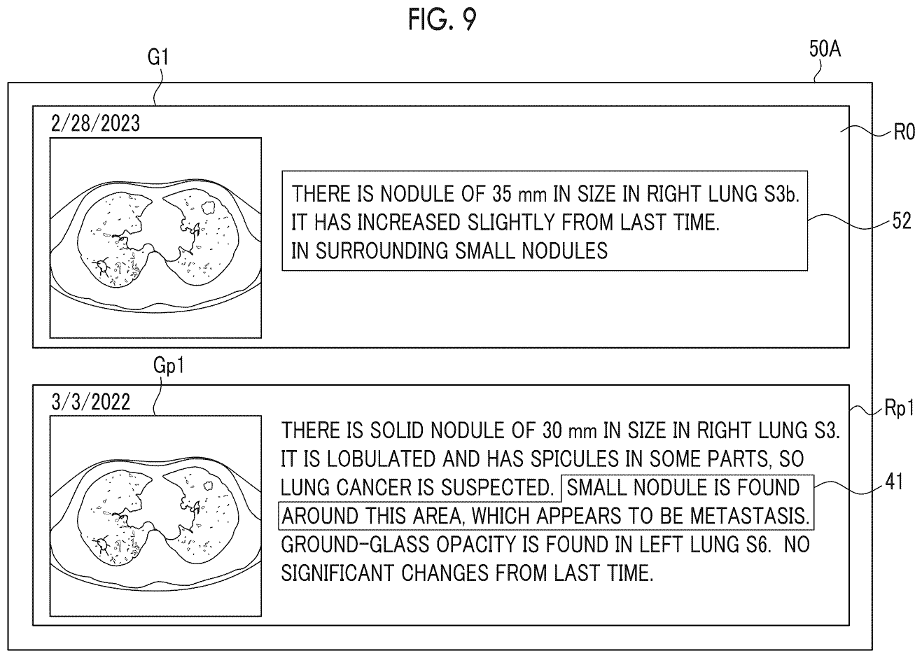

FIG. 9 is a diagram showing an interpretation report creation screen displayed on the interpretation WS 3 in the second embodiment. As shown in FIG. 9, on an interpretation report creation screen 50A, the medical image G1 to be interpreted is displayed above the past interpretation report Rp1, and a report region 52 is displayed on the right side thereof. The radiologist of the interpretation WS 3 interprets the medical image G1 and describes an interpretation report in the report region 52.

In the second embodiment, each time the conversion of the sentence input to the report region 52 into Japanese is confirmed, the description specifying unit 22 analyzes the current interpretation report and past interpretation reports Rpi that have been input into the report region 52 so far, and thereby specifies corresponding descriptions included in each of the currently being created interpretation report R0 and the past interpretation reports Rpi, as in the first embodiment.

Here, as shown in FIG. 9, at the time when the conversion of the first sentence “There is a nodule of 35 mm in size in right lung S3b” is confirmed, and at the time when the second sentence “It has increased slightly from last time” is confirmed, the description specifying unit 22 specifies corresponding descriptions included in each of the interpretation report R0 currently being created and the past interpretation report Rp1. In a case in which the first sentence “There is a nodule of 35 mm in size in right lung S3b” is confirmed and the corresponding description is specified, the display controller 23 displays the sentence 40 (see FIG. 3) in the past interpretation report Rpi is displayed in a manner different from other sentences other than the sentence 40, that is, highlights it. In addition, in a case in which the second sentence “It has increased slightly from last time” is confirmed and the corresponding description is specified, the sentence 40 in the past interpretation report Rpi is continuously highlighted. Further, as shown in FIG. 9, it is assumed that the conversion is confirmed with “in the surrounding small nodules”. In this case as well, the description specifying unit 22 specifies the corresponding description in the past interpretation report Rp1, and the display controller 23 highlights the sentence 41 in the past interpretation report Rpi as shown in FIG. 9.

In this way, in the second embodiment, the description of the past interpretation report Rpi corresponding to the description of the interpretation report R0 being created is displayed in a manner different from the other descriptions. Therefore, in a case of creating an interpretation report, it is possible to easily refer to medical documents such as a past interpretation report.

Further, in the first and second embodiments, the interpretation report for the CT image is used as the medical image, but the present disclosure is not limited thereto. A current interpretation report R1 and a past interpretation report Rpi for the medical images acquired by different modalities may be used. This will be described below as a third embodiment.

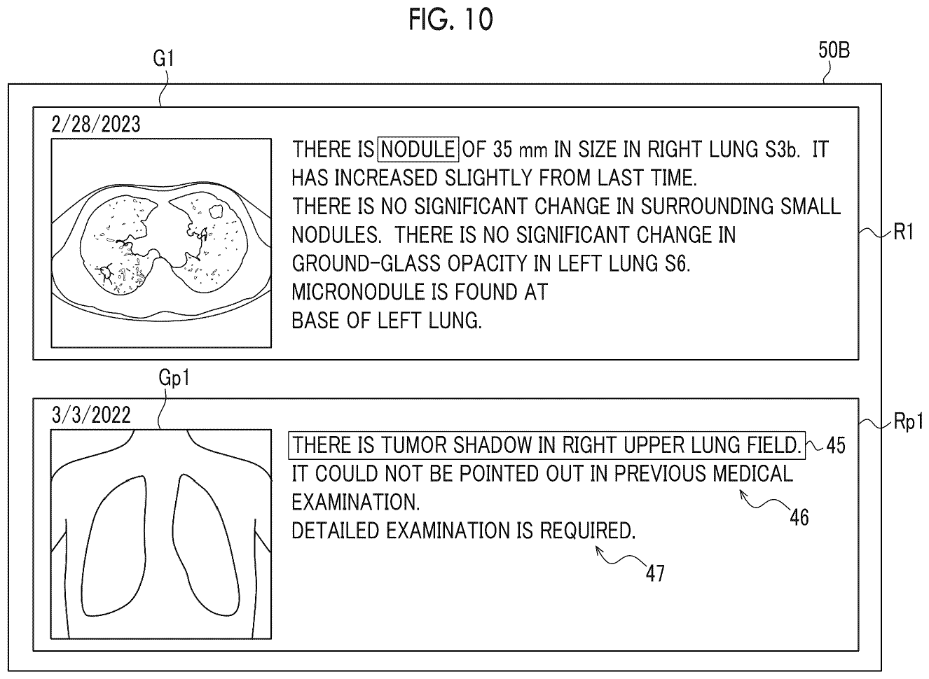

FIG. 10 is a diagram showing a display screen of an interpretation report in the third embodiment. As shown in FIG. 10, the current interpretation report R1 and the past interpretation report Rp1 are displayed on a display screen 50B of the interpretation report. The creation date and time of the current interpretation report R1 is Feb. 28, 2023, and the creation date and time of the past interpretation report Rp1 is Mar. 3, 2022. Furthermore, the current interpretation report R1 and the past interpretation report Rp1 each include medical images G1 and Gp1 for which the interpretation report is created. Note that the medical image G1 is a CT image, and the medical image Gp1 is an X-ray image.

The current interpretation report R1 has the same description content as in the first embodiment. In the past interpretation report Rp1, “There is a tumor shadow in the right upper lung field. It could not be pointed out in the previous medical examination. Detailed examination is required” are described. These three sentences are respectively referred to as sentences 45 to 47.

In the third embodiment, the description specifying unit 22 structures sentences included in the current interpretation report R1 and the past interpretation report Rpi. Then, the description specifying unit 22 specifics mutually corresponding descriptions by associating sentences regarding the same lesion with respect to the four sentences 30 to 33 structured for the current interpretation report R1 and the three sentences 45 to 47 structured for the past interpretation report Rpi. In the third embodiment, among the three sentences 45 to 47 included in the past interpretation report Rpi, only the sentence 45 includes a description regarding the lesion. The sentence 45 includes the description regarding the right lung, and the sentence 30 in the current interpretation report R1 includes a description about the right lung. Therefore, the description specifying unit 22 specifies the sentence 30 in the current interpretation report R1 and the sentence 45 in the past interpretation report Rpi as corresponding descriptions.

In the third embodiment, as shown in FIG. 10, it is assumed that “nodule” is selected in the current interpretation report R1. The display controller 23 displays, in the past interpretation report Rp, the sentence 45 including a description corresponding to “nodule” in the current interpretation report R1 in a manner different from other sentences other than the sentence 45, that is, highlights it. Note that in the third embodiment, in a case in which the character string included in the sentences 31 to 33 in the current interpretation report R1 is selected, highlighting is not performed because there is no corresponding description in the past interpretation report Rp1.

In this way, in the third embodiment, even in a case in which the interpretation reports for the medical images acquired by different modalities are displayed, the description corresponding to the selected character string in the current interpretation report R1 can be displayed in a manner different from other descriptions in the past interpretation report Rpi.

Next, a fourth embodiment of the present disclosure will be described. In the first to third embodiments, the description specifying unit 22 specifies mutually corresponding descriptions included in each of the interpretation reports R1 and Rpi. The fourth embodiment differs from the first to third embodiments in that the description specifying unit 22 further specifies a difference between corresponding descriptions.

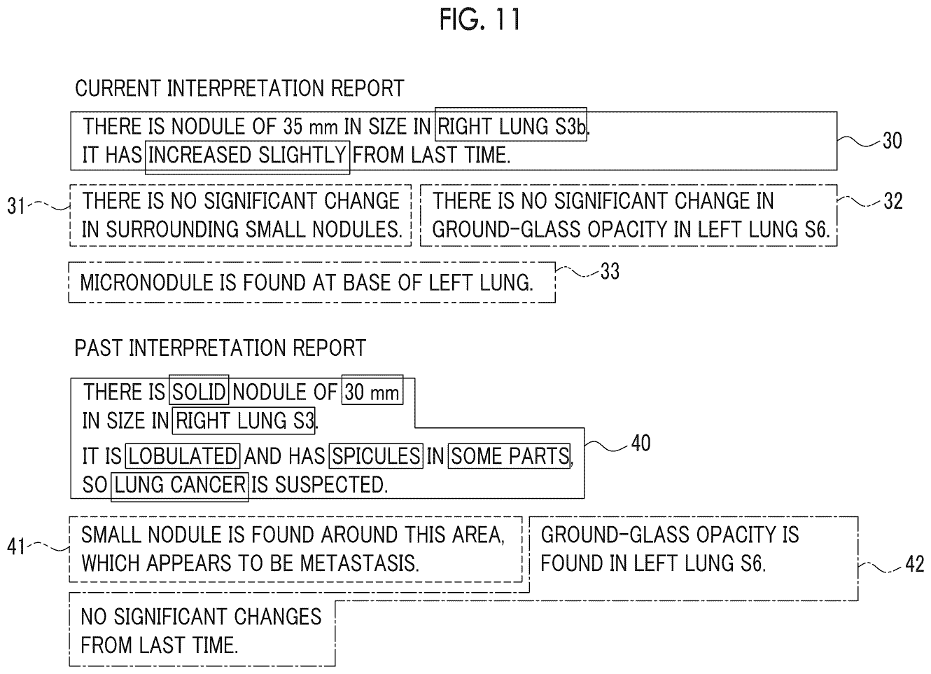

FIG. 11 is a diagram for describing specification of a difference. The descriptions of the current interpretation report R1 and the past interpretation report Rp1 shown in FIG. 11 are the same as in the first embodiment. Note that in the fourth embodiment, since the specification of the mutually corresponding descriptions included in each of the interpretation reports R1 and Rp1 is performed in the same manner as in the first embodiment, the detailed description thereof will be omitted here. In the fourth embodiment, the description specifying unit 22 compares descriptions between sentences to which the description corresponds.

For example, the sentence 30 of the current interpretation report R1 corresponds to the sentence 40 of the past interpretation report Rp1. The description specifying unit 22 compares the sentence 30 “There is a nodule of 35 mm in size in right lung S3b. It has increased slightly from last time” and the sentence 40 “There is a solid nodule of 30 mm in size in right lung S3. It is lobulated and has spicules in some parts, so lung cancer is suspected”, and specifies a difference in the sentence 40 of the past interpretation report Rpi by specifying a different description from the sentence 30 of the current interpretation report R1.

In the fourth embodiment, the description specifying unit 22 specifies, for example, differences in descriptions regarding the property, the size, and the location of a lesion. Therefore, the description specifying unit 22 specifies “right lung S3b” and “increased slightly” as differences for the sentence 30 of the current interpretation report R1. Further, the description specifying unit 22 specifies, for the sentence 40 of the past interpretation report Rpi, “right lung S3”, “30 mm”, “solid”, “lobulated”, “some parts”, “spicules”, and “lung cancer” as differences.

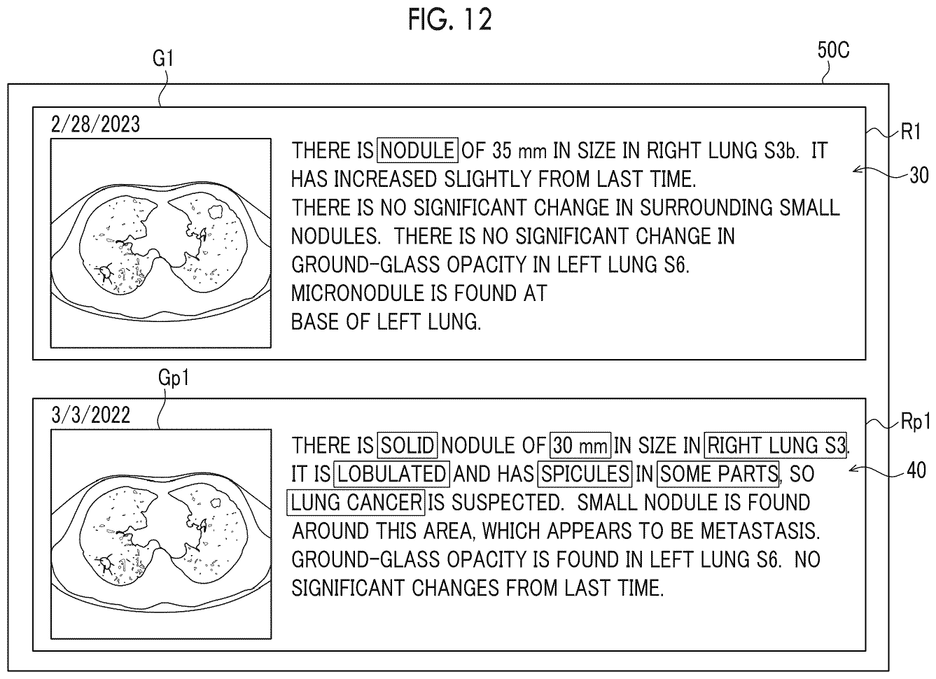

FIG. 12 is a diagram showing a display screen of an interpretation report in the fourth embodiment. In the fourth embodiment, in a case in which the user selects a character string included in the description of the current interpretation report R1 on a display screen 50C of the interpretation report, the display controller 23 displays the difference in the description corresponding to the selected character string in the past interpretation report Rpi in a manner different from the descriptions other than the difference, that is, highlights it. For example, as shown in FIG. 12, it is assumed that the user selects “nodule” described in the current interpretation report R1. The display controller 23 highlights the difference specified by the description specifying unit 22 in the sentence 40 of the past interpretation report Rp1 whose description corresponds to the sentence 30 in which “nodule” is described in the current interpretation report R1. As shown in FIG. 12, in the sentence 40, “right lung S3”, “30 mm”, “solid”, “lobulated”, “some parts”, “spicules”, and “lung cancer” are highlighted. Note that in FIG. 12, the difference is highlighted by surrounding it with a rectangle, but the present disclosure is not limited thereto.

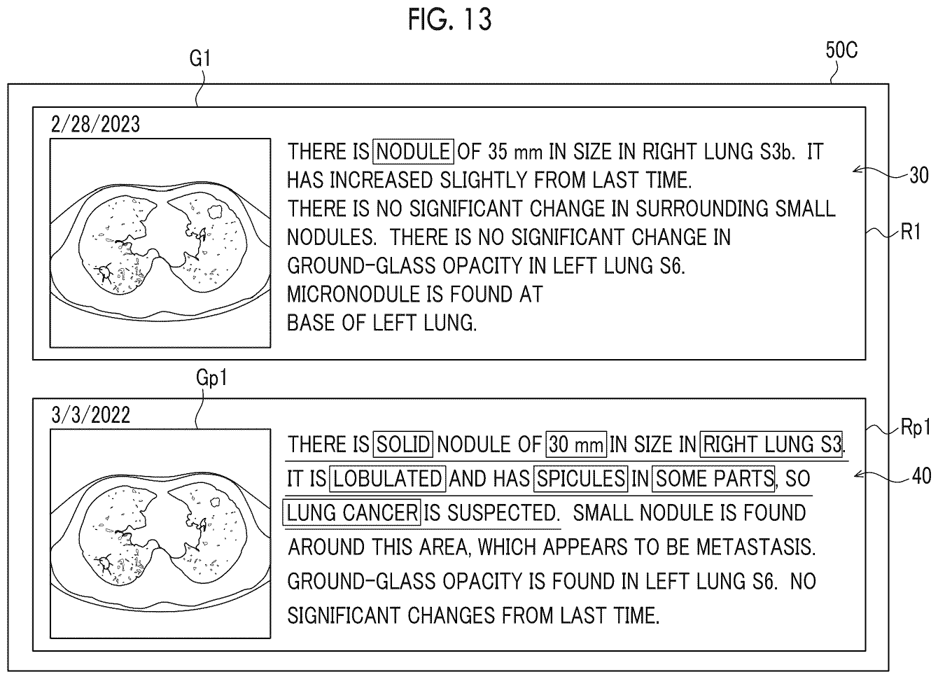

Note that, as shown in FIG. 13, the sentence 40 whose description corresponds to the sentence 30 may be emphasized, and the difference may also be emphasized. In FIG. 13, the sentence 40 is underlined to emphasize the sentence 40, and the difference is emphasized by surrounding it with a rectangle, but the present disclosure is not limited thereto.

In this way, in the fourth embodiment, the designation of a character string for the current interpretation report R1 is received, and a difference in the description of the past interpretation report Rpi corresponding to the description including the designated character string is specified, and the difference is displayed in a manner different from descriptions other than the difference. Therefore, in a case of comparing the current interpretation report R1 and the past interpretation report Rpi, it is possible to easily recognize the difference in the mutually corresponding descriptions.

Note that, in each of the above embodiments, the current interpretation report R1 and one past interpretation report Rpi are displayed, but the present disclosure is not limited thereto. In a case in which there are a plurality of past interpretation reports Rpi, corresponding descriptions between the current interpretation report R1 and each of the plurality of past interpretation reports Rpi may be specified. In this case, in a case in which a plurality of past interpretation reports Rpi are displayed in addition to the current interpretation report R1 and a character string is designated in the current interpretation report R1, a description corresponding to a description including a character string in each of the plurality of past interpretation reports Rpi may be displayed in a manner different from other descriptions.

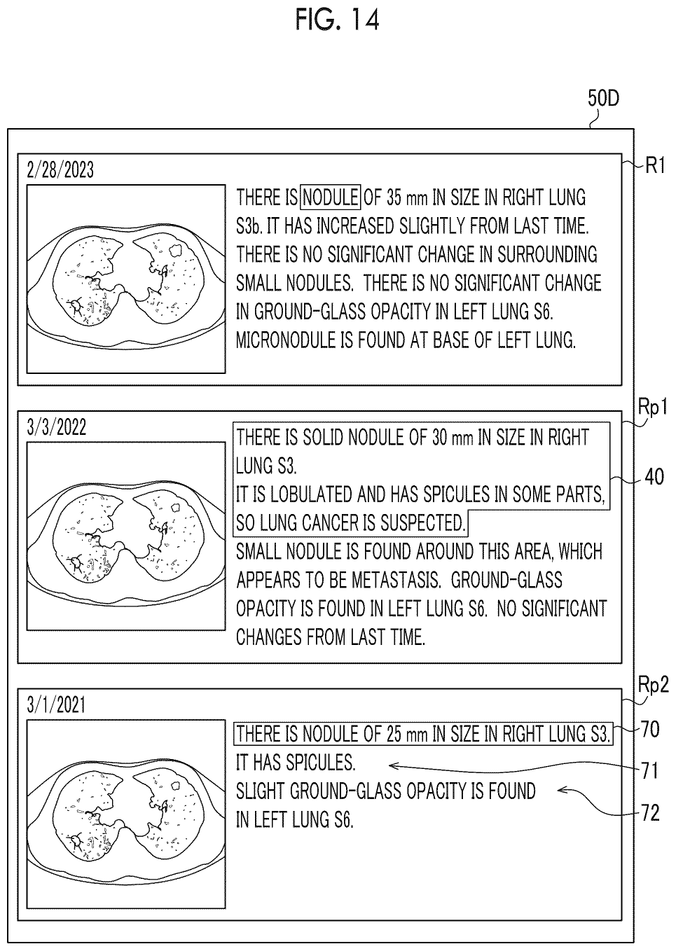

FIG. 14 is a diagram showing a display screen of an interpretation report in which two past interpretation reports Rp1 and Rp2 are displayed in addition to the current interpretation report R1. The descriptions of the current interpretation report R1 and the past interpretation report Rp1 displayed on a display screen 50D are the same as in the first embodiment. The description of the past interpretation report Rp2 includes three sentences 70 to 72 of “There is a nodule of 25 mm in size in right lung S3. It has spicules. A slight ground-glass opacity is found in left lung S6”. The three sentences 70 to 72 of the past interpretation report Rp2 correspond in description to the sentences 30 to 32 of the current interpretation report R1, respectively.

Note that, on the display screen 50D of the interpretation report shown in FIG. 14, the past interpretation report Rp1 whose creation time is closer to the current interpretation report R1 is displayed higher than the past interpretation report Rp2. Therefore, in the past interpretation report Rp1 whose creation time is closer to the current interpretation report R1, display of a different character string, that is, highlighting is performed with priority over the past interpretation report Rp2.

It is assumed that “nodule” of the current interpretation report R1 is selected as a character string on the display screen 50D of the interpretation report shown in FIG. 14. In this case, the display controller 23 displays the sentence 40 corresponding to the sentence 30 in the past interpretation report Rp1 in a manner different from other sentences other than sentence 40, that is, highlights it, and displays the sentence 70 corresponding to the sentence 30 in the past interpretation report Rp2 in a manner different from other sentences other than the sentence 70, that is, highlights it. Thereby, even in a case in which a plurality of past interpretation reports Rpi are displayed, it is possible to easily recognize a description corresponding to the description of the current interpretation report R1.

Note that the sentence 40 of only the past interpretation report Rp1 whose creation time is closest to the current interpretation report R1 may be displayed in a manner different from other sentences than the sentence 40, that is, highlighted, and in response to an instruction from the user, the sentence 70 corresponding to the sentence 30 in the past interpretation report Rp2 may be highlighted. This also causes the character strings of the past interpretation report Rp1 whose creation time is closer to the current interpretation report R1 to be highlighted with priority over the past interpretation report Rp2.

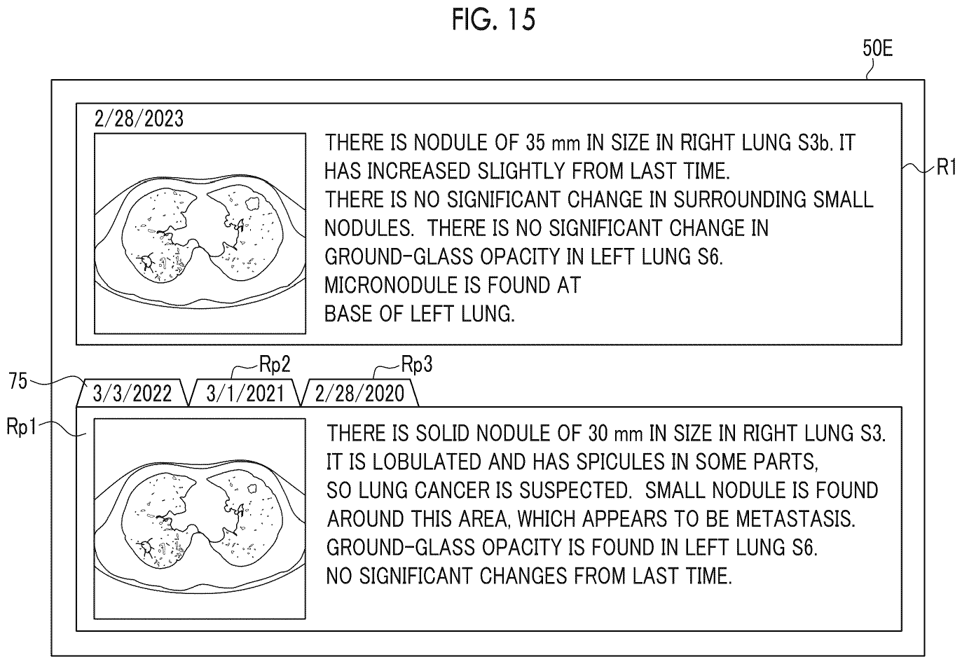

Further, the plurality of past interpretation reports Rpi are not limited to being displayed at the same time. As shown in a display screen 50E of FIG. 15, a plurality of past interpretation reports Rpi may be displayed so as to be selectable using tabs 75 in which their creation dates and times are described. In this case, the past interpretation report Rp1 whose creation time is closer to the current interpretation report R1 may be placed in the tab on the left so that it is displayed before the past interpretation report Rp2.

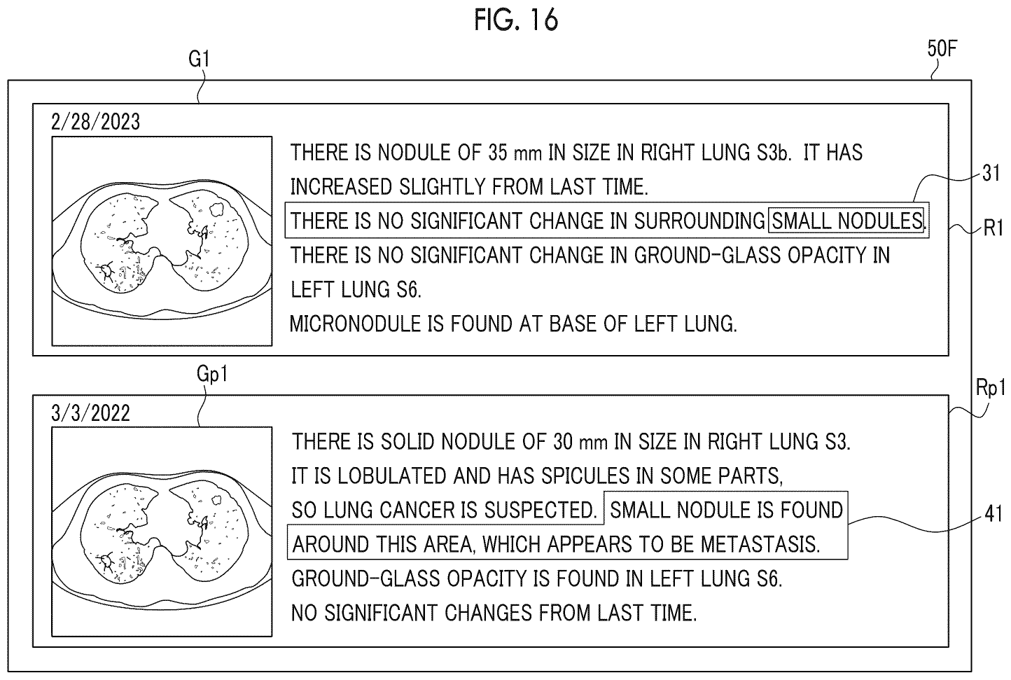

Further, in each of the above embodiments, in a case in which a character string is designated in the current interpretation report R1, the description of the past interpretation report Rpi corresponding to the description of the current interpretation report R1 including a character string is displayed in a manner different from other descriptions. In addition to this, as shown in FIG. 16, the description in the current interpretation report R1 including the designated character string may be displayed in a manner different from other descriptions. That is, as shown in a display screen 50F of FIG. 16, in a case in which “small nodules” is selected in the current interpretation report R1, in addition to highlighting the sentence 41 whose description corresponds to the sentence 31 in which “small nodules” is described, the sentence 31 in which “small nodules” is described may also be highlighted.

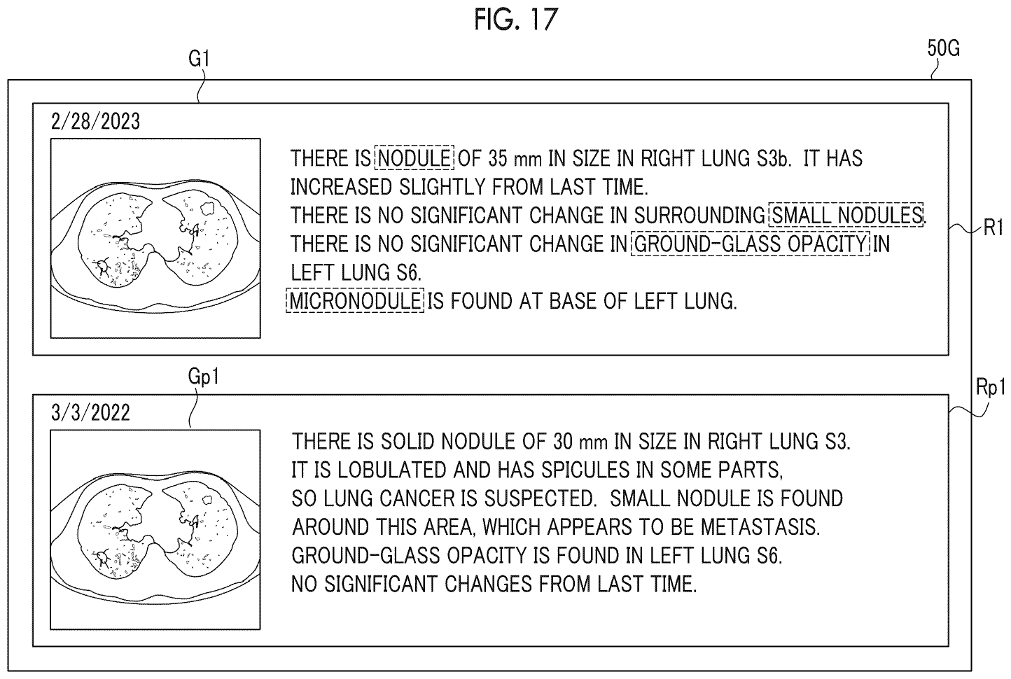

In addition, as shown in a display screen 50G of FIG. 17, in the current interpretation report R1, a character string related to a description that mutually corresponds to the past interpretation report Rpi may be displayed in a manner different from other character strings other than the related character strings, that is, highlighted. In FIG. 17, the character string is highlighted by surrounding it with a broken-line rectangle, but the present disclosure is not limited thereto. Thereby, the user can easily designate the corresponding description in the current interpretation report R1.

In addition, in each of the above embodiments, the current interpretation report R1 and the past interpretation report Rpi are displayed at the same time, but the present disclosure is not limited thereto. First, in a case in which only the current interpretation report R1 is displayed and the user selects a character string in the current interpretation report R1, the past interpretation report Rpi may be displayed, and in the displayed past interpretation reports Rpi, sentences to which the description corresponds may be displayed in a manner different from other sentences, that is, highlighted.

Further, in each of the above embodiments, the technology of the present disclosure is applied in the case of creating an interpretation report using a medical image with the lung as the diagnosis target, but the diagnosis target is not limited to the lung. In addition to the lung, any part of a human body such as a heart, liver, brain, and limbs can be diagnosed. In this case, the diagnostic guideline according to the diagnosis target part may be acquired, and the corresponding portion corresponding to the item of the diagnostic guideline in the interpretation report may be specified.

In each of the above embodiments, for example, as hardware structures of processing units that execute various kinds of processing, such as the acquisition unit 21, the description specifying unit 22, and the display controller 23, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different types of two or more processors (for example, a combination of a plurality of FPGAs or a combination of the CPU and the FPGA). In addition, a plurality of processing units may be configured by one processor.

As an example where a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

The supplementary notes of the present disclosure will be described below.

### Supplementary Note 1

An information processing apparatus comprising at least one processor,
wherein the processor is configured to:
   acquire a plurality of medical documents having different creation times;
   specify mutually corresponding descriptions in each of one medical document among the plurality of medical documents and at least one other medical document other than the one medical document;
   display the one medical document on a display;
   receive designation of a character string for the one medical document; and
   display a description of the at least one other medical document corresponding to a description including the designated character string in a manner different from other descriptions other than the description corresponding to the description including the designated character string.

### Supplementary Note 2

The information processing apparatus according to Supplementary Note 1,
wherein the processor is configured to:
   classify descriptions included in each of the plurality of medical documents for each lesion; and
   specify the mutually corresponding descriptions for each lesion.

### Supplementary Note 3

The information processing apparatus according to Supplementary Note 1 or 2,
wherein the processor is configured to:
   further specify a difference between corresponding descriptions included in each of the plurality of medical documents; and
   display the difference in a manner different from descriptions other than the difference in the at least one other medical document, instead of the description corresponding to the description including the designated character string or in addition to the description corresponding to the description including the designated character string.

### Supplementary Note 4

The information processing apparatus according to any one of Supplementary Notes 1 to 3,
wherein the medical document includes a medical image corresponding to the medical document, and
the processor is configured to display at least a partial region of the medical image related to the differently displayed description in a manner different from other regions other than the partial region.

### Supplementary Note 5

The information processing apparatus according to any one of Supplementary Notes 1 to 4,
wherein the processor is configured to display character strings related to the mutually corresponding descriptions in the one medical document in a manner different from other character strings other than the related character strings.

### Supplementary Note 6

The information processing apparatus according to any one of Supplementary Notes 1 to 5,
wherein the processor is configured to display the description including the character string in the one medical document in a manner different from other descriptions other than the description including the character string.

### Supplementary Note 7

The information processing apparatus according to any one of Supplementary Notes 1 to 6,
wherein the one medical document is a medical document currently being created.

### Supplementary Note 8

The information processing apparatus according to any one of Supplementary Notes 1 to 7,
wherein the plurality of medical documents include medical documents acquired by different modalities at least in part.

### Supplementary Note 9

The information processing apparatus according to any one of Supplementary Notes 1 to 8,
wherein the processor is configured to, in a case in which a plurality of the other medical documents are displayed, preferentially display the description in the different manner for the other medical document whose creation time is closer to the one medical document.

### Supplementary Note 10

An information processing method executed by a computer, the information processing method comprising:
acquiring a plurality of medical documents having different creation times;
specifying mutually corresponding descriptions in each of one medical document among the plurality of medical documents and at least one other medical document other than the one medical document;
displaying the one medical document on a display;
receiving designation of a character string for the one medical document; and
displaying a description of the at least one other medical document corresponding to a description including the designated character string in a manner different from other descriptions other than the description corresponding to the description including the designated character string.

### Supplementary Note 11

An information processing program causing a computer to execute:
acquiring a plurality of medical documents having different creation times;
specifying mutually corresponding descriptions in each of one medical document among the plurality of medical documents and at least one other medical document other than the one medical document;
displaying the one medical document on a display;
receiving designation of a character string for the one medical document; and
displaying a description of the at least one other medical document corresponding to a description including the designated character string in a manner different from other descriptions other than the description corresponding to the description including the designated character string.

What is claimed is:

1. An information processing apparatus comprising at least one processor,
wherein the processor is configured to:
acquire a plurality of medical documents having different creation times;
specify mutually corresponding descriptions in each of one medical document among the plurality of medical documents and at least one other medical document other than the one medical document;
display the one medical document on a display;
receive designation of a character string for the one medical document; and
display a description of the at least one other medical document corresponding to a description including the designated character string in a manner different from other descriptions other than the description corresponding to the description including the designated character string.

2. The information processing apparatus according to claim 1,
wherein the processor is configured to:
classify descriptions included in each of the plurality of medical documents for each lesion; and
specify the mutually corresponding descriptions for each lesion.

3. The information processing apparatus according to claim 1,
wherein the processor is configured to:
further specify a difference between corresponding descriptions included in each of the plurality of medical documents; and
display the difference in a manner different from descriptions other than the difference in the at least one other medical document, instead of the description corresponding to the description including the designated character string or in addition to the description corresponding to the description including the designated character string.

4. The information processing apparatus according to claim 1,
wherein the medical document includes a medical image corresponding to the medical document, and
the processor is configured to display at least a partial region of the medical image related to the differently displayed description in a manner different from other regions other than the partial region.

5. The information processing apparatus according to claim 1,
wherein the processor is configured to display character strings related to the mutually corresponding descriptions in the one medical document in a manner different from other character strings other than the related character strings.

6. The information processing apparatus according to claim 1,
wherein the processor is configured to display the description including the character string in the one medical document in a manner different from other descriptions other than the description including the character string.

7. The information processing apparatus according to claim 1,
wherein the one medical document is a medical document currently being created.

8. The information processing apparatus according to claim 1,
wherein the plurality of medical documents include medical documents acquired by different modalities at least in part.

9. The information processing apparatus according to claim 1,
wherein the processor is configured to, in a case in which a plurality of the other medical documents are displayed, preferentially display the description in the different manner for the other medical document whose creation time is closer to the one medical document.

10. An information processing method executed by a computer, the information processing method comprising:
acquiring a plurality of medical documents having different creation times;
specifying mutually corresponding descriptions in each of one medical document among the plurality of medical documents and at least one other medical document other than the one medical document;
displaying the one medical document on a display;
receiving designation of a character string for the one medical document; and
displaying a description of the at least one other medical document corresponding to a description including the designated character string in a manner different from other descriptions other than the description corresponding to the description including the designated character string.

11. A non-transitory computer-readable storage medium that stores an information processing program causing a computer to execute:
acquiring a plurality of medical documents having different creation times;
specifying mutually corresponding descriptions in each of one medical document among the plurality of medical documents and at least one other medical document other than the one medical document;
displaying the one medical document on a display;
receiving designation of a character string for the one medical document; and
displaying a description of the at least one other medical document corresponding to a description including the designated character string in a manner different from other descriptions other than the description corresponding to the description including the designated character string.
